(19) United States
(12) Patent Application Publication
(10) Pub. No.: US 2023/0257706 A1
(43) Pub. Date: Aug. 17, 2023

(54) T lymphocyte and use thereof

(71) Applicant: FAPON BIOTHERAPY INC., Shenzhen, Guangdong (CN)

(72) Inventors: Baolei WANG, Shenzhen, Guangdong (CN); Xiaolong DU, Shenzhen, Guangdong (CN); Liang PENG, Shenzhen, Guangdong (CN); Xianjin WANG, Shenzhen, Guangdong (CN); Lijun YE, Shenzhen, Guangdong (CN)

(21) Appl. No.: 18/014,479

(22) PCT Filed: Jul. 6, 2021
(86) PCT No.: PCT/CN2021/104802

§ 371 Date: Jan. 5, 2023

(30) Foreign Application Priority Data
Jul. 7, 2020 (CN) 202010648451.1

(51) Int. Cl. C12N 5/0783; C07K 16/28; C07K 14/55; C12N 15/62; C12N 15/86; C12N 5/10

(52) U.S. Cl. CPC C12N 5/0636; C07K 14/55; C07K 16/2815; C12N 5/10; C12N 15/62; C12N 15/86; C07K 2317/53; C07K 2319/03; C12N 2740/15021

(57) ABSTRACT

Provided is a T lymphocyte. The T lymphocyte co-expresses a fusion protein and a chimeric antigen receptor, and the chimeric antigen receptor identifies a tumor antigen, herein the chimeric antigen receptor includes: an extracellular region; a transmembrane region, herein the transmembrane region is connected to the extracellular region, and embedded into a cell membrane of a transgenic lymphocyte; and an intracellular region, herein the intracellular region is connected to the transmembrane region, and the intracellular region includes an immune co-stimulatory molecule intracellular segment. The fusion protein includes: an immune checkpoint single-chain antibody and a T cell activation molecule.

### CROSS-REFERENCE TO RELATED APPLICATION

The present application is a National Stage of International Patent Application No. PCT/CN2021/104802, filed on Jul. 06, 2021, and claims priority to and interest of patent application No. 202010648451.1, filed to the China National Intellectual Property Administration on Jul. 7, 2020, the disclosure of which is hereby incorporated by reference in its entirety.

### SEQUENCE LISTING

The instant application contains a Sequence Listing which has submitted electronically in ASCII format and is hereby incorporated by reference in its entirety. Said ASCII copy is named PN199119 SEQ LIST.txt and is 25,231 bytes in size. The sequence listing contains 11 sequences, which is identical in substance to the sequences disclosed in the PCT application, and includes no new matter.

### TECHNICAL FIELD

The present disclosure relates to the field of biopharmaceuticals, specifically, the present disclosure relates to a T lymphocyte and a use thereof, and more specifically, the present disclosure relates to a T lymphocyte, a lentivirus, a transgenic lymphocyte, a construct, a method for preparing the T lymphocyte or the transgenic lymphocyte, a therapeutic composition for treating a cancer, and a method for reducing expression of an immune checkpoint on the surface of the T lymphocyte.

### BACKGROUND

Chimeric antigen receptor T cell immunotherapy, referred to as a CAR-T technology, is a method to transform patient’s T cells in vitro, make the patient’s T cells have an ability to recognize tumor cells, and then transfuse them back into the patient’s body for treatment after expanded culture in vitro. At present, CAR-T with CD19 as a target site makes a great achievement in treatment of B cell blood tumors. However, according to clinical research results, the efficacy of CD19 CAR-T in treatment of B cell lymphoma is far less than that in treatment of B cell acute lymphoblastic leukemia (B-ALL). This may be because the B cell lymphoma is a solid tumor, and its cell surface expresses a large number of PD-L1 molecules. Although there is no literature reporting the expression level of PD-L1 on B cells of the patient with B-ALL at present, it is found from clinical researches on diffuse large B cell lymphoma that the expression level of PD-L1 on the surface of the patient’s B cell lymphoma is directly related to the clinical efficacy, and the patient with the low expression of PD-L1 on the surface of the B cell lymphoma has the higher survival rates in combination therapy, chemotherapy alone and PD-1 antibody immunotherapy. At the same time, during treatment of other target solid tumors with the CAR-T technology, the tumor cells may also escape the killing of CAR-T by overexpressing PD-L1, it is suggested that during the treatment of the solid tumors, the blocking of a PD-1/PD-L1 signal pathway may maximize the clinical efficacy of CAR-T, and the patient is benefited.

IL-21 is generated by a CD4T cell and a natural killer T (NKT) cell, stimulates the maturation of a CD8T cell and a natural killer (NK) cell and enhances its cytotoxicity, as well as has the functions of promoting the differentiation of the memory CD8T cell and the like. The many effects of IL-21 make it become a potential target site of the immunotherapy. However, since IL-21R is widely expressed in the T cell, the B cell, the NK cell and a bone marrow cell, how to make IL-21 specifically act on the CAR-T cell to control its toxicity becomes the focus of the cell immunotherapy. In addition, the half-life of IL-21 itself is very short, and the effective transformation is also needed to improve the half-life.

### SUMMARY

The present disclosure aims to at least solve one of technical problems in related technologies to a certain extent.

The inventor develops a CART that secretes a fusion protein of a PD-1 antibody and an IL-21, PD-1 is mainly expressed on the surface of a T cell, and it is mainly a CD8+T cell. The secreted fusion protein of a PD-1 antibody and an IL-21 selectively binds to the surface of the T cell and the CART cell. On the one hand, it blocks a PD-1/PD-L1 signal pathway, and on the other hand, it makes IL-21 specifically act on the T cell and the CART cell, thereby exercising its dual functions; at the same time, the fusion protein greatly improves the half-life of a drug due to the increased molecular weight.

In view of this, in a first aspect of the present disclosure, the present disclosure provides a T lymphocyte. According to an embodiment of the present disclosure, the T lymphocyte co-expresses a fusion protein and a chimeric antigen receptor, herein the chimeric antigen receptor includes: an extracellular region, herein the extracellular region includes a heavy chain variable region, a light chain variable region and a CD8 hinge region of a single chain antibody, and the single-chain antibody specifically recognizes a tumor antigen; a transmembrane region, herein the transmembrane region is connected with the extracellular region, the transmembrane region includes a transmembrane segment of CD8 and is embedded into a cell membrane of the T lymphocyte; and an intracellular region, herein the intracellular region is connected with the transmembrane region, and the intracellular region includes an intracellular segment of 4-1BB and a CD3 ζ chain; the fusion protein includes: an immune checkpoint single-chain antibody and a T cell activation molecule. The T lymphocyte according to the embodiment of the present disclosure secretes the fusion protein including the immune checkpoint single-chain antibody and the T cell activation molecule, specifically acts on the CAR-T cell with the double advantages of the immune checkpoint single-chain antibody and the T cell activation molecule, reduces the inhibition of a tumor microenvironment on CART, and makes the CART cell have a more long-term effect; at the same time, the inventor is surprised to find that the CART cell according to the embodiment of the present disclosure expresses a very low proportion of immune checkpoint molecules on the cell surface, and has a stronger tumor killing effect; and the CART cell according to the embodiment of the present disclosure, compared with the CART cell that secretes the immune checkpoint single-chain antibody or the T cell activation molecule separately, greatly improves the binding specificity of the T cell activation molecule to the T lymphocyte, and reduces the toxicity of the drug.

According to an embodiment of the present disclosure, the above T lymphocyte may further include at least one of the following additional technical features:

According to an embodiment of the present disclosure, the immune checkpoint includes at least one selected from PD-1, PD-L1, CTLA-4, TIM3, LAG3, BTLA and TIGIT.

According to an embodiment of the present disclosure, the T cell activation molecule includes at least one selected from IL2, IL7, IL9, IL12, IL15, IL18 and IL21.

According to an embodiment of the present disclosure, the immune checkpoint is PD-1, and the T cell activation molecule is IL21.

According to an embodiment of the present disclosure, the C terminal of the IL21 is connected with the N terminal of a PD-1 single-chain antibody; preferably, the C terminal of the PD-1 single-chain antibody is connected with the N terminal of the IL21. The inventor finds that while the C terminal of the PD-1 single-chain antibody is connected with the N terminal of the IL21, the expression quantity of PD-1 on the surface of the T lymphocyte is lower, and the killing effect of the T lymphocyte on tumors is more significant.

According to an embodiment of the present disclosure, the fusion protein further includes a linker peptide, and the linker peptide is arranged between the immune checkpoint single-chain antibody and the T cell activation molecule.

According to an embodiment of the present disclosure, the linker peptide has an amino acid sequence shown in SEQ ID NO: 1.

GGGGSGGGGSGGGGS (SEQ ID NO: 1).

According to an embodiment of the present disclosure, the N terminal of the linker peptide is connected with the C terminal of the immune checkpoint single-chain antibody, and the C terminal of the linker peptide is connected with the N terminal of the T cell activation molecule.

According to an embodiment of the present disclosure, the fusion protein has an amino acid sequence shown in SEQ ID NO: 2 or SEQ ID NO: 3.

EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRGGGGSGGGGSGGGGSQVQLVESGGGVVQPGRSLRLDCKASGI
TFSNSGMHWVRQAPGKGLEWVAVIWYDGSKRYYADSVKGRFTISRDNSKN
TLFLQMNSLRAEDTAVYYCATNDDYWGQGTLVTVSSGGGGSGGGGSGGGG
SMHKSSSQGQDRHMIRMRQLIDIVDQLKNYVNDLVPEFLPAPEDVETNCE
WSAFSCFQKAQLKSANTGNNERIINVSIKKLKRKPPSTNAGRRQKHRLTC
PSCDSYEKKPPKEFLERFKSLLQKMIHQHLSSRTHGSEDS (SEQ ID N
O:2).

HKSSSQGQDRHMIRMRQLIDIVDQLKNYVNDLVPEFLPAPEDVETNCEWS
AFSCFQKAQLKSANTGNNERIINVSIKKLKRKPPSTNAGRRQKHRLTCPS
CDSYEKKPPKEFLERFKSLLQKMIHQHLSSRTHGSEDSGGGGSGGGGSGG
GGSMEIVLTQSPATLSLSPGERATLSCRASQSVSSYLAWYQQKPGQAPRL
LIYDASNRATGIPARFSGSGSGTDFTLTISSLEPEDFAVYYCQQSSNWPR
TFGQGTKVEIKRGGGGSGGGGSGGGGSQVQLVESGGGVVQPGRSLRLDCK
ASGITFSNSGMHWVRQAPGKGLEWVAVIWYDGSKRYYADSVKGRFTISRD
NSKNTLFLQMNSLRAEDTAVYYCATNDDYWGQGTLVTVSS (SEQ ID N
O:3).

Herein, the fusion protein scFV-IL21 has the amino acid sequence shown in SEQ ID NO: 2, and the fusion protein IL21-scFV has the amino acid sequence shown in SEQ ID NO: 3, herein scFV represents the immune checkpoint single-chain antibody, IL21 represents that the T cell activation molecule is IL21, the connection sequence of scFV and IL21 in the fusion protein scFV-IL21 is that the C terminal of the immune checkpoint single-chain antibody is connected with the N terminal of IL21, and the connection sequence of scFV and IL21 in the fusion protein IL21-scFV is that the C terminal of IL21 is connected with the N terminal of the immune checkpoint single-chain antibody.

In a second aspect of the present disclosure, the present disclosure provides a lentivirus. According to an embodiment of the present disclosure, the lentivirus carries the following nucleic acid molecules: (a) a nucleic acid molecule encoding a fusion protein, herein the fusion protein includes: the immune checkpoint single-chain antibody and the T cell activation molecule; and (b) a nucleic acid molecule encoding a chimeric antigen receptor, herein an extracellular region of the chimeric antigen receptor recognizes a tumor antigen. The lentivirus according to the embodiment of the present disclosure is introduced into the T lymphocyte of a receptor cell, which may express and secrete the fusion protein including the immune checkpoint single-chain antibody and the T cell activation molecule as well as the chimeric antigen receptor in the receptor cell, reduce the expression of the immune checkpoint on the surface of the T lymphocyte, and reduce the inhibition of the tumor microenvironment on the T lymphocyte, thus the killing effect of the T cell on the tumors is more effective and long-term, and the safety is higher.

According to an embodiment of the present disclosure, the above lentivirus may further include at least one of the following additional technical features.

According to an embodiment of the present disclosure, the immune checkpoint is PD-1, the T cell activation molecule is IL21, and the fusion protein has the amino acid sequence shown in SEQ ID NO: 2 or 3.

According to an embodiment of the present disclosure, the tumor antigen is CD19, and the chimeric antigen receptor has an amino acid sequence shown in SEQ ID NO: 4.

DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELRVKFSRSADAPAYKQGQNQLYNELNLGRREEYDVLDKRRGRDPEMGG
KPRRKNPQEGLYNELQKDKMAEAYSEIGMKGERRRGKGHDGLYQGLSTAT
KDTYDALHMQALPPR (SEQ ID NO: 4).

According to an embodiment of the present disclosure, the nucleic acid molecule encoding the fusion protein has any one of nucleotide sequences shown in SEQ ID NOs: 5 or 6.

GAGATCGTGCTGACCCAGTCTCCAGCCACACTGAGCCTGTCTCCTGGCGA
GAGAGCCACCCTGTCTTGTAGGGCCAGCCAGTCCGTGAGCTCTTACCTGG
CCTGGTATCAGCAGAAGCCAGGCCAGGCCCCAAGACTGCTGATCTACGAC
GCCTCCAACAGAGCCACCGGCATCCCAGCCAGATTTTCTGGCTCCGGCTC
TGGCACCGACTTCACACTGACCATCAGCTCTCTGGAGCCAGAGGATTTCG
CCGTGTATTACTGCCAGCAGAGCTCTAACTGGCCAAGAACATTCGGGCAG
GGGACCAAGGTGGAAATCAAGAGGGGCGGCGGCGGCTCTGGCGGCGGCGG
CTCCGGCGGCGGCGGCTCTCAGGTGCAGCTGGTGGAGAGCGGCGGCGGAG
TGGTGCAGCCAGGCAGATCTCTGAGACTGGATTGCAAGGCCAGCGGCATC
ACCTTCAGCAATTCCGGCATGCACTGGGTGCGGCAGGCCCCCGGCAAGGG
CCTGGAGTGGGTGGCCGTGATCTGGTATGACGGCTCTAAGCGGTACTATG
CCGACTCTGTGAAGGGCAGATTCACCATCTCCAGGGACAACTCCAAGAAT
ACCCTGTTCCTGCAGATGAACAGCCTGAGGGCCGAGGATACCGCCGTGTA
CTATTGCGCCACCAACGACGATTACTGGGGCCAGGGCACACTGGTGACCG
TGTCCAGCGGCGGCGGCGGCTCTGGCGGCGGCGGCTCCGGCGGCGGCGGC
TCTATGCACAAATCAAGCTCCCAAGGTCAAGATCGCCACATGATTAGAAT
GCGTCAACTTATAGATATTGTTGATCAGCTGAAAAATTATGTGAATGACT
TGGTCCCTGAATTTCTGCCAGCTCCAGAAGATGTAGAGACAAACTGTGAG
TGGTCAGCTTTTTCCTGTTTTCAGAAGGCCCAACTAAAGTCAGCAAATAC
AGGAAACAATGAAAGGATAATCAATGTATCAATTAAAAAGCTGAAGAGGA
AACCACCTTCCACAAATGCAGGGAGAAGACAGAAACACAGACTAACATGC
CCTTCATGTGATTCTTATGAGAAAAAACCACCCAAAGAATTCCTAGAAAG
ATTCAAATCACTTCTCCAAAAGATGATTCATCAGCATCTGTCCTCTAGAA
CACACGGAAGTGAAGATTCC (SEQ ID NO: 5).

CACAAATCAAGCTCCCAAGGTCAAGATCGCCACATGATTAGAATGCGTCA
ACTTATAGATATTGTTGATCAGCTGAAAAATTATGTGAATGACTTGGTCC
CTGAATTTCTGCCAGCTCCAGAAGATGTAGAGACAAACTGTGAGTGGTCA
GCTTTTTCCTGTTTTCAGAAGGCCCAACTAAAGTCAGCAAATACAGGAAA
CAATGAAAGGATAATCAATGTATCAATTAAAAAGCTGAAGAGGAAACCAC
CTTCCACAAATGCAGGGAGAAGACAGAAACACAGACTAACATGCCCTTCA
TGTGATTCTTATGAGAAAAAACCACCCAAAGAATTCCTAGAAAGATTCAA
ATCACTTCTCCAAAAGATGATTCATCAGCATCTGTCCTCTAGAACACACG
GAAGTGAAGATTCCGGTGGCGGTGGCTCGGGCGGTGGTGGGTCGGGTGGC
GGCGGATCTATGGAGATCGTGCTGACCCAGTCTCCAGCCACACTGAGCCT
GTCTCCTGGCGAGAGAGCCACCCTGTCTTGTAGGGCCAGCCAGTCCGTGA
GCTCTTACCTGGCCTGGTATCAGCAGAAGCCAGGCCAGGCCCCAAGACTG
CTGATCTACGACGCCTCCAACAGAGCCACCGGCATCCCAGCCAGATTTTC
TGGCTCCGGCTCTGGCACCGACTTCACACTGACCATCAGCTCTCTGGAGC
CAGAGGATTTCGCCGTGTATTACTGCCAGCAGAGCTCTAACTGGCCAAGA
ACATTCGGGCAGGGGACCAAGGTGGAAATCAAGAGGGGCGGCGGCGGCTC
TGGCGGCGGCGGCTCCGGCGGCGGCGGCTCTCAGGTGCAGCTGGTGGAGA
GCGGCGGCGGAGTGGTGCAGCCAGGCAGATCTCTGAGACTGGATTGCAAG
GCCAGCGGCATCACCTTCAGCAATTCCGGCATGCACTGGGTGCGGCAGGC
CCCCGGCAAGGGCCTGGAGTGGGTGGCCGTGATCTGGTATGACGGCTCTA
AGCGGTACTATGCCGACTCTGTGAAGGGCAGATTCACCATCTCCAGGGAC
AACTCCAAGAATACCCTGTTCCTGCAGATGAACAGCCTGAGGGCCGAGGA
TACCGCCGTGTACTATTGCGCCACCAACGACGATTACTGGGGCCAGGGCA
CACTGGTGACCGTGTCCAGC (SEQ ID NO: 6).

Herein, the fusion protein encoded by the nucleic acid molecule with the nucleotide sequence shown in SEQ ID NO: 5 has the amino acid sequence shown in SEQ ID NO: 2, and the fusion protein encoded by the nucleic acid molecule with the nucleotide sequence shown in SEQ ID NO: 6 has the amino acid sequence shown in SEQ ID NO: 3.

According to an embodiment of the present disclosure, the nucleic acid molecule encoding the chimeric antigen receptor has a nucleotide sequence shown in SEQ ID NO: 7.

GACATCCAGATGACACAGACTACATCCTCCCTGTCTGCCTCTCTGGGAGA
CAGAGTCACCATCAGTTGCAGGGCAAGTCAGGACATTAGTAAATATTTAA
ATTGGTATCAGCAGAAACCAGATGGAACTGTTAAACTCCTGATCTACCAT
ACATCAAGATTACACTCAGGAGTCCCATCAAGGTTCAGTGGCAGTGGGTC
TGGAACAGATTATTCTCTCACCATTAGCAACCTGGAGCAAGAAGATATTG
CCACTTACTTTTGCCAACAGGGTAATACGCTTCCGTACACGTTCGGAGGG
GGGACCAAGCTGGAGATCACAGGTGGCGGTGGCTCGGGCGGTGGTGGGTC
GGGTGGCGGCGGATCTGAGGTGAAACTGCAGGAGTCAGGACCTGGCCTGG
TGGCGCCCTCACAGAGCCTGTCCGTCACATGCACTGTCTCAGGGGTCTCA
TTACCCGACTATGGTGTAAGCTGGATTCGCCAGCCTCCACGAAAGGGTCT
GGAGTGGCTGGGAGTAATATGGGGTAGTGAAACCACATACTATAATTCAG
CTCTCAAATCCAGACTGACCATCATCAAGGACAACTCCAAGAGCCAAGTT
TTCTTAAAAATGAACAGTCTGCAAACTGATGACACAGCCATTTACTACTG
TGCCAAACATTATTACTACGGTGGTAGCTATGCTATGGACTACTGGGGCC
AAGGAACCTCAGTCACCGTCTCCTCAACCACGACGCCAGCGCCGCGACCA
CCAACACCGGCGCCCACCATCGCGTCGCAGCCCCTGTCCCTGCGCCCAGA
GGCGTGCCGGCCAGCGGCGGGGGGCGCAGTGCACACGAGGGGGCTGGACT
TCGCCTGTGATATCTACATCTGGGCGCCCTTGGCCGGGACTTGTGGGGTC
CTTCTCCTGTCACTGGTTATCACCCTTTACTGCAAACGGGGCAGAAAGAA
ACTCCTGTATATATTCAAACAACCATTTATGAGACCAGTACAAACTACTC
AAGAGGAAGATGGCTGTAGCTGCCGATTTCCAGAAGAAGAAGAAGGAGGA
TGTGAACTGAGAGTGAAGTTCAGCAGGAGCGCAGACGCCCCCGCGTACAA
GCAGGGCCAGAACCAGCTCTATAACGAGCTCAATCTAGGACGAAGAGAGG
AGTACGATGTTTTGGACAAGAGACGTGGCCGGGACCCTGAGATGGGGGGA
AAGCCGAGAAGGAAGAACCCTCAGGAAGGCCTGTACAATGAACTGCAGAA
AGATAAGATGGCGGAGGCCTACAGTGAGATTGGGATGAAAGGCGAGCGCC
GGAGGGGCAAGGGGCACGATGGCCTTTACCAGGGTCTCAGTACAGCCACC
AAGGACACCTACGACGCCCTTCACATGCAGGCCCTGCCCCCTCGC (SEQ
ID NO: 7).

In a third aspect of the present disclosure, the present disclosure provides a lentivirus. According to an embodiment of the present disclosure, the lentivirus carries a nucleic acid molecule with a nucleotide sequence shown in SEQ ID NO: 8 or 9.

GACATCCAGATGACACAGACTACATCCTCCCTGTCTGCCTCTCTGGGAGA
CAGAGTCACCATCAGTTGCAGGGCAAGTCAGGACATTAGTAAATATTTAA
ATTGGTATCAGCAGAAACCAGATGGAACTGTTAAACTCCTGATCTACCAT
ACATCAAGATTACACTCAGGAGTCCCATCAAGGTTCAGTGGCAGTGGGTC
TGGAACAGATTATTCTCTCACCATTAGCAACCTGGAGCAAGAAGATATTG
CCACTTACTTTTGCCAACAGGGTAATACGCTTCCGTACACGTTCGGAGGG
GGGACCAAGCTGGAGATCACAGGTGGCGGTGGCTCGGGCGGTGGTGGGTC
GGGTGGCGGCGGATCTGAGGTGAAACTGCAGGAGTCAGGACCTGGCCTGG
TGGCGCCCTCACAGAGCCTGTCCGTCACATGCACTGTCTCAGGGGTCTCA
TTACCCGACTATGGTGTAAGCTGGATTCGCCAGCCTCCACGAAAGGGTCT
GGAGTGGCTGGGAGTAATATGGGGTAGTGAAACCACATACTATAATTCAG
CTCTCAAATCCAGACTGACCATCATCAAGGACAACTCCAAGAGCCAAGTT
TTCTTAAAAATGAACAGTCTGCAAACTGATGACACAGCCATTTACTACTG
TGCCAAACATTATTACTACGGTGGTAGCTATGCTATGGACTACTGGGGCC
AAGGAACCTCAGTCACCGTCTCCTCAACCACGACGCCAGCGCCGCGACCA
CCAACACCGGCGCCCACCATCGCGTCGCAGCCCCTGTCCCTGCGCCCAGA
GGCGTGCCGGCCAGCGGCGGGGGGCGCAGTGCACACGAGGGGGCTGGACT
TCGCCTGTGATATCTACATCTGGGCGCCCTTGGCCGGGACTTGTGGGGTC
CTTCTCCTGTCACTGGTTATCACCCTTTACTGCAAACGGGGCAGAAAGAA
ACTCCTGTATATATTCAAACAACCATTTATGAGACCAGTACAAACTACTC
AAGAGGAAGATGGCTGTAGCTGCCGATTTCCAGAAGAAGAAGAAGGAGGA
TGTGAACTGAGAGTGAAGTTCAGCAGGAGCGCAGACGCCCCCGCGTACAA
GCAGGGCCAGAACCAGCTCTATAACGAGCTCAATCTAGGACGAAGAGAGG
AGTACGATGTTTTGGACAAGAGACGTGGCCGGGACCCTGAGATGGGGGGA
AAGCCGAGAAGGAAGAACCCTCAGGAAGGCCTGTACAATGAACTGCAGAA
AGATAAGATGGCGGAGGCCTACAGTGAGATTGGGATGAAAGGCGAGCGCC
GGAGGGGCAAGGGGCACGATGGCCTTTACCAGGGTCTCAGTACAGCCACC
AAGGACACCTACGACGCCCTTCACATGCAGGCCCTGCCCCCTCGCGCCAC
TAACTTCTCCCTGTTGAAACAAGCAGGGGATGTCGAAGAGAATCCCGGGC
CAGAGATCGTGCTGACCCAGTCTCCAGCCACACTGAGCCTGTCTCCTGGC
GAGAGAGCCACCCTGTCTTGTAGGGCCAGCCAGTCCGTGAGCTCTTACCT
GGCCTGGTATCAGCAGAAGCCAGGCCAGGCCCCAAGACTGCTGATCTACG
ACGCCTCCAACAGAGCCACCGGCATCCCAGCCAGATTTTCTGGCTCCGGC
TCTGGCACCGACTTCACACTGACCATCAGCTCTCTGGAGCCAGAGGATTT
CGCCGTGTATTACTGCCAGCAGAGCTCTAACTGGCCAAGAACATTCGGGC
AGGGGACCAAGGTGGAAATCAAGAGGGGCGGCGGCGGCTCTGGCGGCGGC
GGCTCCGGCGGCGGCGGCTCTCAGGTGCAGCTGGTGGAGAGCGGCGGCGG
AGTGGTGCAGCCAGGCAGATCTCTGAGACTGGATTGCAAGGCCAGCGGCA
TCACCTTCAGCAATTCCGGCATGCACTGGGTGCGGCAGGCCCCCGGCAAG
GGCCTGGAGTGGGTGGCCGTGATCTGGTATGACGGCTCTAAGCGGTACTA
TGCCGACTCTGTGAAGGGCAGATTCACCATCTCCAGGGACAACTCCAAGA
ATACCCTGTTCCTGCAGATGAACAGCCTGAGGGCCGAGGATACCGCCGTG
TACTATTGCGCCACCAACGACGATTACTGGGGCCAGGGCACACTGGTGAC
CGTGTCCAGCGGCGGCGGCGGCTCTGGCGGCGGCGGCTCCGGCGGCGGCG
GCTCTATGCACAAATCAAGCTCCCAAGGTCAAGATCGCCACATGATTAGA
ATGCGTCAACTTATAGATATTGTTGATCAGCTGAAAAATTATGTGAATGA
CTTGGTCCCTGAATTTCTGCCAGCTCCAGAAGATGTAGAGACAAACTGTG
AGTGGTCAGCTTTTTCCTGTTTTCAGAAGGCCCAACTAAAGTCAGCAAAT
ACAGGAAACAATGAAAGGATAATCAATGTATCAATTAAAAAGCTGAAGAG
GAAACCACCTTCCACAAATGCAGGGAGAAGACAGAAACACAGACTAACAT
GCCCTTCATGTGATTCTTATGAGAAAAAACCACCCAAAGAATTCCTAGAA
AGATTCAAATCACTTCTCCAAAAGATGATTCATCAGCATCTGTCCTCTAG
AACACACGGAAGTGAAGATTCC (SEQ ID NO: 8).

GACATCCAGATGACACAGACTACATCCTCCCTGTCTGCCTCTCTGGGAGA
CAGAGTCACCATCAGTTGCAGGGCAAGTCAGGACATTAGTAAATATTTAA
ATTGGTATCAGCAGAAACCAGATGGAACTGTTAAACTCCTGATCTACCAT
ACATCAAGATTACACTCAGGAGTCCCATCAAGGTTCAGTGGCAGTGGGTC
TGGAACAGATTATTCTCTCACCATTAGCAACCTGGAGCAAGAAGATATTG
CCACTTACTTTTGCCAACAGGGTAATACGCTTCCGTACACGTTCGGAGGG
GGGACCAAGCTGGAGATCACAGGTGGCGGTGGCTCGGGCGGTGGTGGGTC
GGGTGGCGGCGGATCTGAGGTGAAACTGCAGGAGTCAGGACCTGGCCTGG
TGGCGCCCTCACAGAGCCTGTCCGTCACATGCACTGTCTCAGGGGTCTCA
TTACCCGACTATGGTGTAAGCTGGATTCGCCAGCCTCCACGAAAGGGTCT
GGAGTGGCTGGGAGTAATATGGGGTAGTGAAACCACATACTATAATTCAG
CTCTCAAATCCAGACTGACCATCATCAAGGACAACTCCAAGAGCCAAGTT
TTCTTAAAAATGAACAGTCTGCAAACTGATGACACAGCCATTTACTACTG
TGCCAAACATTATTACTACGGTGGTAGCTATGCTATGGACTACTGGGGCC
AAGGAACCTCAGTCACCGTCTCCTCAACCACGACGCCAGCGCCGCGACCA
CCAACACCGGCGCCCACCATCGCGTCGCAGCCCCTGTCCCTGCGCCCAGA
GGCGTGCCGGCCAGCGGCGGGGGGCGCAGTGCACACGAGGGGGCTGGACT
TCGCCTGTGATATCTACATCTGGGCGCCCTTGGCCGGGACTTGTGGGGTC
CTTCTCCTGTCACTGGTTATCACCCTTTACTGCAAACGGGGCAGAAAGAA
ACTCCTGTATATATTCAAACAACCATTTATGAGACCAGTACAAACTACTC
AAGAGGAAGATGGCTGTAGCTGCCGATTTCCAGAAGAAGAAGAAGGAGGA
TGTGAACTGAGAGTGAAGTTCAGCAGGAGCGCAGACGCCCCCGCGTACAA
GCAGGGCCAGAACCAGCTCTATAACGAGCTCAATCTAGGACGAAGAGAGG
AGTACGATGTTTTGGACAAGAGACGTGGCCGGGACCCTGAGATGGGGGGA
AAGCCGAGAAGGAAGAACCCTCAGGAAGGCCTGTACAATGAACTGCAGAA
AGATAAGATGGCGGAGGCCTACAGTGAGATTGGGATGAAAGGCGAGCGCC
GGAGGGGCAAGGGGCACGATGGCCTTTACCAGGGTCTCAGTACAGCCACC
AAGGACACCTACGACGCCCTTCACATGCAGGCCCTGCCCCCTCGCGCCAC
TAACTTCTCCCTGTTGAAACAAGCAGGGGATGTCGAAGAGAATCCCGGGC
CACACAAATCAAGCTCCCAAGGTCAAGATCGCCACATGATTAGAATGCGT
CAACTTATAGATATTGTTGATCAGCTGAAAAATTATGTGAATGACTTGGT
CCCTGAATTTCTGCCAGCTCCAGAAGATGTAGAGACAAACTGTGAGTGGT
CAGCTTTTTCCTGTTTTCAGAAGGCCCAACTAAAGTCAGCAAATACAGGA
AACAATGAAAGGATAATCAATGTATCAATTAAAAAGCTGAAGAGGAAACC
ACCTTCCACAAATGCAGGGAGAAGACAGAAACACAGACTAACATGCCCTT
CATGTGATTCTTATGAGAAAAAACCACCCAAAGAATTCCTAGAAAGATTC
AAATCACTTCTCCAAAAGATGATTCATCAGCATCTGTCCTCTAGAACACA
CGGAAGTGAAGATTCCGGTGGCGGTGGCTCGGGCGGTGGTGGGTCGGGTG
GCGGCGGATCTATGGAGATCGTGCTGACCCAGTCTCCAGCCACACTGAGC
CTGTCTCCTGGCGAGAGAGCCACCCTGTCTTGTAGGGCCAGCCAGTCCGT
GAGCTCTTACCTGGCCTGGTATCAGCAGAAGCCAGGCCAGGCCCCAAGAC
TGCTGATCTACGACGCCTCCAACAGAGCCACCGGCATCCCAGCCAGATTT
TCTGGCTCCGGCTCTGGCACCGACTTCACACTGACCATCAGCTCTCTGGA
GCCAGAGGATTTCGCCGTGTATTACTGCCAGCAGAGCTCTAACTGGCCAA
GAACATTCGGGCAGGGGACCAAGGTGGAAATCAAGAGGGGCGGCGGCGGC
TCTGGCGGCGGCGGCTCCGGCGGCGGCGGCTCTCAGGTGCAGCTGGTGGA
GAGCGGCGGCGGAGTGGTGCAGCCAGGCAGATCTCTGAGACTGGATTGCA
AGGCCAGCGGCATCACCTTCAGCAATTCCGGCATGCACTGGGTGCGGCAG
GCCCCCGGCAAGGGCCTGGAGTGGGTGGCCGTGATCTGGTATGACGGCTC
TAAGCGGTACTATGCCGACTCTGTGAAGGGCAGATTCACCATCTCCAGGG
ACAACTCCAAGAATACCCTGTTCCTGCAGATGAACAGCCTGAGGGCCGAG
GATACCGCCGTGTACTATTGCGCCACCAACGACGATTACTGGGGCCAGGG
CACACTGGTGACCGTGTCCAGC(SEQ ID NO: 9).

Herein, the nucleic acid molecule with the nucleotide sequence shown in SEQ ID NO: 8 encodes CD19CAR-scFV-IL21, and the nucleic acid molecule with the nucleotide sequence shown in SEQ ID NO: 9 encodes CD19CAR-IL21-scFV.

In a fourth aspect of the present disclosure, the present disclosure provides a transgenic lymphocyte. According to an embodiment of the present disclosure, the transgenic lymphocyte co-expresses a fusion protein and a chimeric antigen receptor, and the chimeric antigen receptor recognizes a tumor antigen, herein the chimeric antigen receptor includes: an extracellular region; a transmembrane region, herein the transmembrane region is connected with the extracellular region, and embedded into a cell membrane of the transgenic lymphocyte; and an intracellular region, herein the intracellular region is connected with the transmembrane region, and the intracellular region includes an intracellular segment of an immune co-stimulatory molecule. The fusion protein includes: an immune checkpoint single-chain antibody and a T cell activation molecule. The transgenic lymphocyte according to the embodiment of the present disclosure expresses the chimeric antigen receptor and secretes the fusion protein including the immune checkpoint single-chain antibody and the T cell activation molecule, the double advantages of the immune checkpoint single-chain antibody and the T cell activation molecule specifically acts on the transgenic lymphocyte, the inhibition of a tumor microenvironment on the transgenic lymphocyte is reduced, and the effect of the transgenic lymphocyte is more long-term; at the same time, the inventor is surprised to find that the transgenic lymphocyte according to the embodiment of the present disclosure expresses a very low proportion of immune checkpoint molecules on the cell surface, and has a stronger tumor killing effect; and the transgenic lymphocyte according to the embodiment of the present disclosure, compared with the transgenic lymphocyte that secretes the immune checkpoint single-chain antibody or the T cell activation molecule separately, greatly improves the binding specificity of the T cell activation molecule to the T lymphocyte, and reduces the toxicity of the drug.

According to an embodiment of the present disclosure, the above transgenic lymphocyte may further include at least one of the following additional technical features.

According to an embodiment of the present disclosure, the intracellular segment of the immune co-stimulatory molecule is independently selected from at least one of 4-1 BB, OX-40, CD40L, CD27, CD30, CD28, CD3 and derivatives thereof.

According to an embodiment of the present disclosure, the intracellular segment of the immune co-stimulatory molecule is an intracellular segment of 4-1BB and CD3.

According to an embodiment of the present disclosure, the lymphocyte is a CD3⁺T lymphocyte.

According to an embodiment of the present disclosure, the lymphocyte is a CD8⁺T lymphocyte.

According to an embodiment of the present disclosure, the lymphocyte is a natural killer cell.

According to an embodiment of the present disclosure, the lymphocyte is a natural killer T cell.

According to an embodiment of the present disclosure, the immune checkpoint includes at least one selected from PD-1, PD-L1, CTLA-4, TIM3, LAG3, BTLA and TIGIT.

According to an embodiment of the present disclosure, the T cell activation molecule includes at least one selected from IL2, IL7, IL9, IL12, IL15, IL18 and IL21.

According to an embodiment of the present disclosure, the immune checkpoint is PD-1, and the T cell activation molecule is IL21.

According to an embodiment of the present disclosure, the C terminal of the IL21 is connected with the N terminal of the PD-1 single-chain antibody.

According to an embodiment of the present disclosure, the C terminal of the PD-1 single-chain antibody is connected with the N terminal of the IL21. Furthermore, the expression of PD-1 on the surface of the transgenic lymphocyte according to the embodiment of the present disclosure is lower, and the killing effect of the transgenic lymphocyte on the tumor cells is more significant.

According to an embodiment of the present disclosure, the fusion protein further includes a linker peptide, and the linker peptide is arranged between the immune checkpoint single-chain antibody and the T cell activation molecule.

According to an embodiment of the present disclosure, the linker peptide has the amino acid sequence shown in SEQ ID NO: 1.

According to an embodiment of the present disclosure, the N terminal of the linker peptide is connected with the C terminal of the immune checkpoint single-chain antibody, and the C terminal of the linker peptide is connected with the N terminal of the T cell activation molecule.

According to an embodiment of the present disclosure, the fusion protein has the amino acid sequence shown in SEQ ID NO: 2 or SEQ ID NO: 3.

In a fifth aspect of the present disclosure, the present disclosure provides a construct. According to an embodiment of the present disclosure, the construct includes: a first nucleic acid molecule, the first nucleic acid molecule encodes a fusion protein, and the fusion protein includes: an immune checkpoint single-chain antibody and a T cell activation molecule; and a second nucleic acid molecule, the second nucleic acid molecule encodes a chimeric antigen receptor, and the chimeric antigen receptor recognizes a tumor antigen, herein the fusion protein and the chimeric antigen receptor are as described above. After the construct according to the embodiment of the present disclosure is introduced into the receptor lymphocyte, the chimeric antigen receptor and the fusion protein may be expressed on the surface of the lymphocyte, and the killing effect of the lymphocyte on the tumors is more significant, lasting and safer.

According to an embodiment of the present disclosure, the above construct may further include at least one of the following additional technical features.

According to an embodiment of the present disclosure, the first nucleic acid molecule and the second nucleic acid molecule are set to express the fusion protein and the chimeric antigen receptor in the previously described lymphocyte, and the fusion protein and the chimeric antigen receptor are in a non-fusion form.

According to an embodiment of the present disclosure, the construct further includes: a first promoter, herein the first promoter is operably connected with the first nucleic acid molecule; and a second promoter, herein the second promoter is operably connected with the second nucleic acid molecule. Furthermore, the first nucleic acid molecule and the second nucleic acid molecule may be independently expressed respectively.

According to a specific embodiment of the present disclosure, the first promoter and the second promoter are respectively independently selected from the promoter of U6, H1, CMV, EF-1, LTR or RSV.

According to an embodiment of the present disclosure, the construct further includes: an internal ribosome entry site sequence, the internal ribosome entry site sequence is arranged between the first nucleic acid molecule and the second nucleic acid molecule, and the internal ribosome entry site has a nucleotide sequence shown in SEQ ID NO: 10. Furthermore, the non-fusion form of the expressed fusion protein and the chimeric antigen receptor may be achieved.

GCCCCTCTCCCTCCCCCCCCCCTAACGTTACTGGCCGAAGCCGCTTGGAA
TAAGGCCGGTGTGCGTTTGTCTATATGTTATTTTCCACCATATTGCCGTC
TTTTGGCAATGTGAGGGCCCGGAAACCTGGCCCTGTCTTCTTGACGAGCA
TTCCTAGGGGTCTTTCCCCTCTCGCCAAAGGAATGCAAGGTCTGTTGAAT
GTCGTGAAGGAAGCAGTTCCTCTGGAAGCTTCTTGAAGACAAACAACGTC
TGTAGCGACCCTTTGCAGGCAGCGGAACCCCCCACCTGGCGACAGGTGCC
TCTGCGGCCAAAAGCCACGTGTATAAGATACACCTGCAAAGGCGGCACAA
CCCCAGTGCCACGTTGTGAGTTGGATAGTTGTGGAAAGAGTCAAATGGCT
CTCCTCAAGCGTATTCAACAAGGGGCTGAAGGATGCCCAGAAGGTACCCC
ATTGTATGGGATCTGATCTGGGGCCTCGGTGCACATGCTTTACATGTGTT
TAGTCGAGGTTAAAAAAACGTCTAGGCCCCCCGAACCACGGGGACGTGGT
TTTCCTTTGAAAAACACGATGATAATATGGCCACAACC (SEQ ID NO:
10).

The internal ribosome entry site is usually located in a 5′ untranslated region (UTR) of a ribonucleic acid (RNA) virus genome, so that the translation of one viral protein may be independent of a 5′ cap structure, and the translation of the other protein usually depends on the 5′ cap structure to initiate. The expression of two genes before and after the internal ribosome entry site (IRES) is usually proportional. The introduction of the internal ribosome entry site sequence makes the nucleic acid molecule encoding the chimeric antigen receptor and the nucleic acid molecule encoding the fusion protein independently express respectively.

According to an embodiment of the present disclosure, the construct further includes: a third nucleic acid molecule, the third nucleic acid molecule is arranged between the first nucleic acid molecule and the second nucleic acid molecule, and the third nucleic acid molecule encodes a cleavable linker peptide, the cleavable linker peptide may be cleaved in the lymphocyte. Then the fusion protein and the chimeric antigen receptor expressed in the lymphocyte are cleaved at the linker peptide, so that the chimeric antigen receptor is independently expressed on the surface of the lymphocyte membrane, and the fusion protein is independently separated from the lymphocyte.

According to an embodiment of the present disclosure, the cleavable linker peptide has an amino acid sequence shown in SEQ ID NO: 11.

IDATNFSLLKQAGDVEENPGP (SEQ ID NO: 11).

According to an embodiment of the present disclosure, a vector of the construct is a non-pathogenic virus vector.

According to an embodiment of the present disclosure, the virus vector includes at least one selected from a retrovirus vector, a lentivirus vector and an adenovirus-related virus vector.

In a sixth aspect of the present disclosure, the present disclosure provides a method for preparing the previously described T lymphocyte or the previously described transgenic lymphocyte. According to an embodiment of the present disclosure, the method includes: introducing the previously described construct or the previously described lentivirus into the lymphocyte or the T lymphocyte. The T lymphocyte or the transgenic lymphocyte prepared according to the method of the embodiment of the present disclosure has more significant and lasting killing effect on the tumor cells and the safety is higher.

In a seventh aspect of the present disclosure, the present disclosure provides a therapeutic composition for treating a cancer. According to an embodiment of the present disclosure, the therapeutic composition includes: the previously described construct, the previously described lentivirus, the previously described T lymphocyte or the previously described transgenic lymphocyte. The therapeutic composition according to the embodiment of the present disclosure has the more significant, lasting killing effect on the tumor cells and the safety is higher.

According to an embodiment of the present disclosure, the cancer includes at least one selected from a liver cancer, a pancreatic cancer, an ovarian cancer, a bile duct cancer, a lung cancer, a stomach cancer, an intestinal cancer, an esophageal cancer and a breast cancer.

In an eighth aspect of the present disclosure, the present disclosure provides an application of the previously described T lymphocyte, the previously described lentivirus, the previously described transgenic lymphocyte, the previously described construct or the previously described therapeutic composition in preparation of a drug for treating a cancer.

According to an embodiment of the present disclosure, the cancer includes at least one selected from the liver cancer, the pancreatic cancer, the ovarian cancer, the bile duct cancer, the lung cancer, the stomach cancer, the intestinal cancer, the esophageal cancer and the breast cancer.

In a ninth aspect of the present disclosure, the present disclosure provides a method for treating the cancer. According to an embodiment of the present disclosure, the method includes administering at least one of the followings to a subject suffering from the cancer:
the previously described T lymphocyte;
the T lymphocyte introduced into the previously described lentivirus;
the previously described transgenic lymphocyte;
the T lymphocyte introduced into the previously described construct; and
the previously described therapeutic composition.

According to an embodiment of the present disclosure, the cancer includes at least one selected from the liver cancer, the pancreatic cancer, the ovarian cancer, the bile duct cancer, the lung cancer, the stomach cancer, the intestinal cancer, the esophageal cancer and the breast cancer.

In a tenth aspect of the present disclosure, the present disclosure provides an application of the previously described T lymphocyte, the previously described lentivirus, the previously described transgenic lymphocyte, the previously described construct or the previously described therapeutic composition in treatment of a cancer.

According to an embodiment of the present disclosure, the cancer includes at least one selected from the liver cancer, the pancreatic cancer, the ovarian cancer, the bile duct cancer, the lung cancer, the stomach cancer, the intestinal cancer, the esophageal cancer and the breast cancer.

In an eleventh aspect of the present disclosure, the present disclosure provides a method for reducing expression of an immune checkpoint on the surface of a T lymphocyte. According to an embodiment of the present disclosure, the method includes: making the T lymphocyte express the chimeric antigen receptor and the fusion proteins; or making the T lymphocyte co-culture with a T cell expressing the chimeric antigen receptor and the fusion protein. The fusion protein includes: an immune checkpoint single-chain antibody and a T cell activation molecule, herein the chimeric antigen receptor and the fusion protein are as defined above. The method according to the embodiment of the present disclosure may significantly reduce the expression of the immune checkpoint on the surface of the T lymphocyte, and block an immune escape mechanism.

According to an embodiment of the present disclosure, the immune checkpoint is PD-1, and the T cell activation molecule is IL21.

In conclusion, the innovation points of the present disclosure are as follows.

1) CART is used to secrete the fusion protein of the PD-1 antibody and the IL-21, firstly the PD-1 single-chain antibody selectively binds to the PD-1 molecule on the surface of the T cell, so that IL-21 may also specifically act on the T cell and play its role. Compared with the simple overexpression of IL-21, it reduces the toxicity caused by IL-21 binding to the IL-21 receptor on the surface of other cells.

2) Compared with the expression of the PD-1 antibody and IL-21 alone, the fusion expression of the PD-1 antibody and IL-21 increases the half-life of the CART drug.

3) Experimental data shows that CART secrets the fusion protein of the PD-1 antibody and IL-21, which greatly reduces the proportion of PD-1 expression on the surface of the T cell.

4) Compared with the expression of the PD-1 antibody and IL-21 alone, the fusion expression of the PD-1 antibody and IL-21 shows that CART has the stronger killing effect on the solid tumors.

The additional aspects and advantages of the present disclosure may be partially given in the following descriptions, and some may become apparent from the following descriptions, or it is understood from the practice of the present disclosure.

### BRIEF DESCRIPTION OF THE DRAWINGS

The above and/or additional aspects and advantages of the present disclosure may become apparent and easily understood from descriptions of embodiments in combination with the following drawings, herein:

FIG. 1 is a structure schematic diagram of a nucleic acid constructed in Embodiment 1 of the present disclosure, herein “Hinge” in the figure refers to a hinge region, and “Linker” refers to a linker peptide.

FIG. 2 is a detection result of a CAR19 positive rate of D6 infected with a T cell in Embodiment 3 of the present disclosure.

FIG. 3 is a result diagram of flow sorting monoclonal establishment of a Raji-PD-L1 overexpression target cell line in Embodiment 5 of the present disclosure, herein “Multi-sample” refers to multiple sampling.

FIG. 4 is a result diagram of CART killing a negative control cell K562 in Embodiment 6 of the present disclosure.

FIG. 5 is a result diagram of CART killing RAJI-PD-L1-A3 in Embodiment 6 of the present disclosure.

FIG. 6 is a result diagram of cytokine release in the process of CART killing RAJI-PD-L1-A3 in Embodiment 7 of the present disclosure.

FIG. 7 is a time axis of a CART in vivo efficacy evaluation experiment in Embodiment 8 of the present disclosure.

FIG. 8 is a tumor remission rate after CART in vivo treatment in Embodiment 8 of the present disclosure.

FIG. 9 is a tumor recurrence rate after CART in vivo treatment in Embodiment 8 of the present disclosure.

FIG. 10 is a statistical diagram of tumor remission and recurrence of individual mice in a group after CART in vivo treatment in Embodiment 8 of the present disclosure.

FIG. 11 is a structure schematic diagram of a nucleic acid constructed in Embodiment 9 of the present disclosure, herein “Hinge” in the figure refers to a hinge region, and “Linker” refers to a linker peptide.

FIG. 12 is a detection result of a CAR-MSLN positive rate of D7 infected with a T cell in Embodiment 9 of the present disclosure.

FIG. 13 is detection of IL-21 in an MSLN CAR-T supernatant in Embodiment 10 of the present disclosure.

FIG. 14 is MSLN CAR-T tumor cell killing detection in Embodiment 11 of the present disclosure.

FIG. 15 is IL-2 and IFN-γ secretion detection while MSLN CAR-T and AsPC-1 cells are co-cultured in Embodiment 11 of the present disclosure.

### DETAILED DESCRIPTION OF THE EMBODIMENTS

Embodiments described below with reference to the drawings are exemplary, and are intended to be used to explain the present disclosure, but may not be understood as limitation to the present disclosure.

In addition, terms “first” and “second” are only used for a descriptive purpose, and may not be understood as indicating or implying relative importance or implicitly indicating the number of technical features indicated. Thus, the features defined as the “first” and “second” may explicitly or implicitly include at least one such feature. In the description of the present disclosure, “multiple” means at least two, such as two, and three, unless otherwise specifically defined.

A term “optionally” is used only for the descriptive purpose, and may not be understood as indicating or implying the relative importance. Thus, the feature defined as the “optionally” may explicitly or implicitly include or exclude this feature.

A single-chain antibody (scFv) is a genetic engineering antibody, herein a heavy chain variable region (VH) and a light chain variable region (VL) are linked with a flexible peptide linker. Compared with a Fab region of the whole antibody, the single-chain antibody shows the better tissue penetration pharmacokinetics, and has the complete antigen binding specificity because the antigen binding surface is not changed.

An immune checkpoint is a regulatory molecule that plays an inhibitory role in an immune system, and it is crucial to maintain self tolerance, prevent an autoimmune reaction, and minimize tissue damage by controlling the time and intensity of an immune response and the like. The immune checkpoint is expressed on an immune cell, and inhibits the function of the immune cell, so that a body is unable to generate an effective anti-tumor immune response, and a tumor forms immune escape. Tumor-related immune checkpoint molecules mainly include: PD1, CTLA4, Tim3 and LAG3 and the like.

The “immune checkpoint single-chain antibody” described in the present application is a single-chain antibody with anti-immune checkpoint activity that may specifically bind to the immune checkpoint.

A T lymphocyte activation molecule refers to a molecule that may stimulate the activation or differentiation of a lymphocyte, and enhance the cytotoxicity of a T lymphocyte, such as IL-21.

An immune costimulatory molecule refers to a cell surface molecule and its ligand that provide a costimulatory signal for the complete activation of the T lymphocyte or B lymphocyte, such as 4-1BB, OX-40, CD40L, CD27, CD30, CD28, CD3 and derivatives thereof.

According to a specific embodiment of the present disclosure, the construction of one lentivirus vector is taken as an example. The inventor inserts a target nucleic acid into a virus genome at the position of some virus sequences in order to construct one lentivirus vector, thereby replication defective viruses are generated. In order to generate a virion, the inventor further constructs a packaging cell line (including gag, pol and env genes, but excluding a long terminal repeat (LTR) and a packaging component). The inventor introduces a recombinant plasmid containing the target gene into the packaging cell line together with lentivirus LTR and packaging sequence. The packaging sequence allows a recombinant plasmid RNA transcript to be packaged into a virus particle, and then secreted into a culture medium. The inventor then collects a matrix containing the recombinant lentivirus, selectively concentrates it, and uses it for gene transfer. The lentivirus vector may infect many cell types, including a dividing cell and a non-dividing cell.

In addition, according to an embodiment of the present disclosure, the lentivirus in the embodiment of the present disclosure is a compound lentivirus. In addition to common lentivirus genes gag, pol and env, it also contains other genes with regulatory and structural functions. The lentivirus vector is well-known to those skilled in the art, and the lentivirus includes a human immunodeficiency virus HIV-1 and HIV-2 and a simian immunodeficiency virus SIV. The lentivirus vector is generated by multiple attenuation of a HIV pathogenic gene, for example, deletion of all genes env, vif, vpr, vpu and nef makes the lentivirus vector form a biosafety vector. The recombinant lentivirus vector may infect the non-dividing cell, and may be used for gene transfer and nucleic acid sequence expression in vivo and in vitro at the same time. For example, in a suitable host cell, two or more vectors with packaging functions (gag, pol, env, rev and tat) may infect the non-dividing cell together. The targeting property of the recombinant virus is achieved by the combination of an antibody or a specific ligand (targeting a specific cell type receptor) with a membrane protein. At the same time, the targeting property of the recombinant virus makes the vector have a specific target by inserting an effective sequence (including a regulatory region) into the virus vector, together with another gene encoding a ligand of a receptor on a specific target cell. Various useful lentivirus vectors, as well as vectors generated by various methods and operations, are used to change the expression of cells.

According to an embodiment of the present disclosure, an adeno-associated virus vector (AAV) in the embodiment of the present disclosure may be constructed with a deoxyribonucleic acid (DNA) of one or more well-known serum type adeno-associated virus vectors. In addition, according to an embodiment of the present disclosure, the embodiment of the present disclosure also includes a microgene. The microgene means that combinations (a selected nucleotide sequence and an operable relevant linkage sequence) are used to guide the expression of transformation, transcription and/or gene products in the host cell in vivo or in vitro. The “operable linkage” sequence includes an expression control sequence of a continuous target gene and an expression control sequence acting on a trans or remote control target gene.

In addition, the vector in the embodiment of the present disclosure also includes a conventional control element in cell transfection with the plasmid vector or/and cell infection with the virus vector. A large number of the expression control sequences (including natural, inducible and/or tissue specific promoters) may be used. According to an embodiment of the present disclosure, the promoter is a promoter selected from U6, H1, or a promoter of pol I, pol II and pol III RNA polymerase. According to an embodiment of the present disclosure, the promoter is a tissue specific promoter. According to an embodiment of the present disclosure, the promoter is an inducible promoter. According to an embodiment of the present disclosure, the promoter is a promoter selected from the selected vectors. According to an embodiment of the disclosure, while the lentivirus vector is selected, the promoter is U6, H1, CMV IE gene, EF-1α, ubiquitin C or a glycerophosphate kinase (PGK) promoter. Other conventional expression control sequences include an optional marker or a reporter gene, including a nucleotide sequence encoding geneticin, hygromycin, ampicillin or puromycin drug resistance and the like. Other components of the vector include a replication starting point.

Technologies for constructing the vector are well-known to those skilled in the art, and these technologies include a conventional cloning technology,

According to an embodiment of the present disclosure, the composition in the embodiment of the present disclosure provided to a patient is better applied to biocompatible solution or an acceptable pharmaceutical carrier. Various therapeutic compositions as preparations are suspended or dissolved in pharmaceutically or physiologically acceptable carriers, such as normal saline; and isotonic salt solution or other more apparent formulas of people who are proficient in this way. The appropriate carrier depends to a large extent on the route of administration. Other isotonic sterile injections with and without water and sterile suspensions with and without water are pharmaceutically acceptable carriers.

These methods of expressing and secreting the fusion protein and expressing the specific chimeric antigen receptor targeting antigen CD19 are a part of combination therapy. These virus vectors and anti-tumor T cells for adoptive immunotherapy may be performed alone or in combination with other cancer treatment methods. Under appropriate conditions, one treatment method includes use of one or more drug therapies.

Schemes of the present disclosure are explained below in combination with the embodiments.

Those skilled in the art may understand that the following embodiments are only used to describe the present disclosure, and should not be considered as limiting the scope of the present disclosure. If the specific technologies or conditions are not indicated in the embodiments, it is performed according to the technologies or conditions described in literatures in this field (for example, referring to “Guide to Molecular Cloning Experiment” written by J. Sambrook et al. and translated by Huang Peitang et al., the third edition, Science Press) or according to product specifications. Reagents or instruments used that do not indicate manufacturers are conventional products that may be purchased in the market.

### Embodiment 1: Construction of CART19 (T Cell Expressing Chimeric Antigen Receptor Targeting CD19) Lentivirus Vector Expressing the Fusion Protein of the PD-1 Antibody and IL-21 and its Control Vector

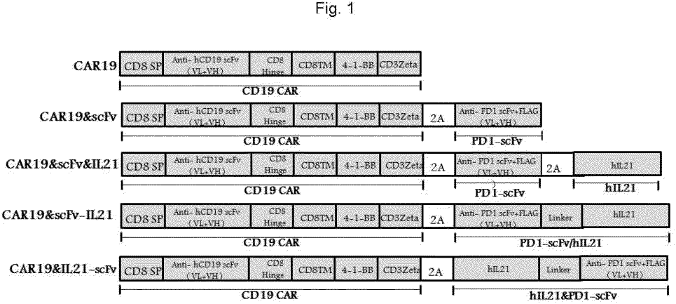

A nucleotide sequence with a structure shown in FIG. 1 was gene-synthesized, and according to a digestion site of a lentivirus vector, nucleotide fragments were constructed onto the lentivirus vector, primers were designed, and the correctness of vector construction was verified by sequencing results.

### Embodiment 2: Packaging and Concentrating of Lentivirus

The 293T was inoculated according to a density of 8×10⁶ cells/150 mm² culture dish, and the state of the cells were observed on the next day. A packaging vector of a third-generation lentivirus was co-transfected to 293T by a polyethylenimine (PEI) transfection method. the solution was changed after 6 hours of the transfection. A Dulbecco’s modified eagle medium (DMEM) containing 10% fetal bovine serum was added according to 15 ml/150 mm² culture dish. The virus supernatant was collected at 48 hours and 72 hours of the transfection and centrifuged at 2000 rpm and 4° C. for 10 min, to remove cell debris, and then impurities were filtered with a 0.45 micron filter, a filtered virus suspension was centrifuged at 25000 rpm and 4° C. for 2 hours, to concentrate the lentivirus, and the concentrated virus was resuspended with an appropriate amount of the culture medium and stored at -80° C.

### Embodiment 3: Generation of CAR-T and Control Cell

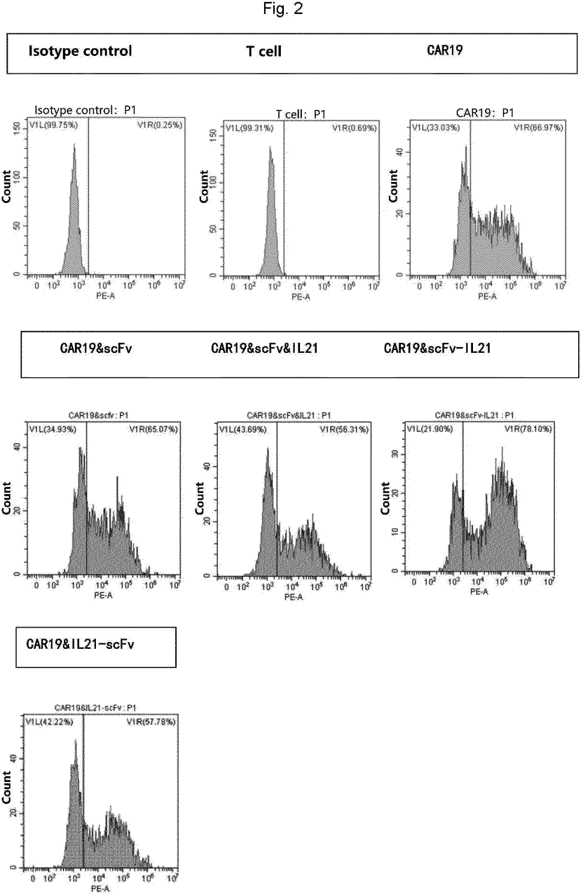

20 mL of blood was drawn, peripheral blood mononuclear cells (PBMC) were separated with Ficall gradient centrifugation, and T cells were separated with the Stemcell T cell negative selection kit (catalog number: 19051). The separated T cells were resuspended to 1×10⁶ cells/ML with the medium containing 5% human AB serum and 300 units/ML IL-2 X-VIVO 15, beads were washed with 1% FBS X-VIVO 15, the pre-washed magnetic beads (Cat#40203D, 10 ML, Life technology) were added according to a proportion of magnetic beads: T cells=2:1, and the T cells were resuspended to 3-5×10⁶ cells/mL with a fresh culture medium after 2-3 days, lentiviruses were added according to a value of MOI=10, and 8 µg/mL of Polybrene was added, after 4-6 hours, the culture medium was supplemented to dilute the cells to 1×10⁶ cells/mL, and replaced with the fresh culture medium on the next day, to maintain the cell concentration at 0.2-0.3×10⁶ PBMC/mL, and after that, the culture medium was replaced every 2-3 days. After 72 hours of virus infection, the cell positive rate was analyzed through a flow cytometry. FIG. 2 is a CAR positive rate detection result on the sixth day (D6) after T cell infection, herein a CAR19 group (66.97%), a CAR19&scFv group (65.07%), a CAR19&scFv&IL21 group (56.31%), a CAR19&scFv-IL21 group (78.1%), and a CAR19&IL21-scFv group (57.78%), it may be seen from results that the CAR19&scFv-IL21 group has the highest infection efficiency.

### Embodiment 4: CART Phenotype Identification

In each group of cells on D6 after infection, a flow cytometry was used to detect its T, B and NK cell populations, and the populations of CD4, CD8 and PD-1 in T cells were detected at the same time. Results were shown in Table 1 (herein, the T cell group represents a normal T cell group, CAR19 represents a T cell group expressing a chimeric antigen receptor targeting CD19, CAR19&scFv represents a CART19 group expressing scFv, CAR19&scFv&IL21 represents a CART19 group expressing scFv and IL21 separately, CAR19&scFv-IL21 represents a CART19 group expressing a fusion protein scFv-IL21, and CAR19&scFv-IL21 represents a CART19 group expressing a fusion protein IL21-scFv), it may be seen that only a small percentage of the T cells (2.87%) in the CAR19&scFv-IL21 group expressed a PD-1 molecule, it is suggested that CAR19&scFv-IL21 may regulate the expression of PD-1 on the surface of the T cells. As a member of the immune checkpoint family, it is reported in literatures that after the T cells are activated by the CD3/CD28 antibody, the expression level of the PD-1 molecule may be up-regulated to inhibit the activation of the T cells. In addition, it is also an important marker of T cell failure.

TABLE 1

| Group | T cell subset | | | | | B cell | NK cell |
|---|---|---|---|---|---|---|---|
| | CD3+ | CD3+/CAR+ | CD3+/CD4+ | CD3+/CD8+ | CD3+/PD-1+ | CD3-/CD19+ | CD3-/CD56+ |
| T cell | 99.11% | 0.69% | 47.40% | 28.50% | 21.60% | 0.00% | 0.20% |
| CAR19 | 99.90% | 66.97% | 50.50% | 27.50% | 50.35% | 0.00% | 0.15% |
| CAR19&scFv | 99.90% | 56.31% | 52.20% | 25.75% | 39.52% | 0.00% | 0.05% |
| CAR19&scFv&IL21 | 99.82% | 65.07% | 49.20% | 25.16% | 20.16% | 0.00% | 0.02% |
| CAR19&scFv-IL21 | 99.72% | 78.10% | 51.75% | 24.75% | 2.87% | 0.00% | 0.10% |
| CAR19&IL21-scFv | 99.61% | 57.78% | 44.60% | 28.70% | 10.36% | 0.00% | 0.20% |

### Embodiment 5: Construction of Raji-PD-L1 Expression Target Cell Line

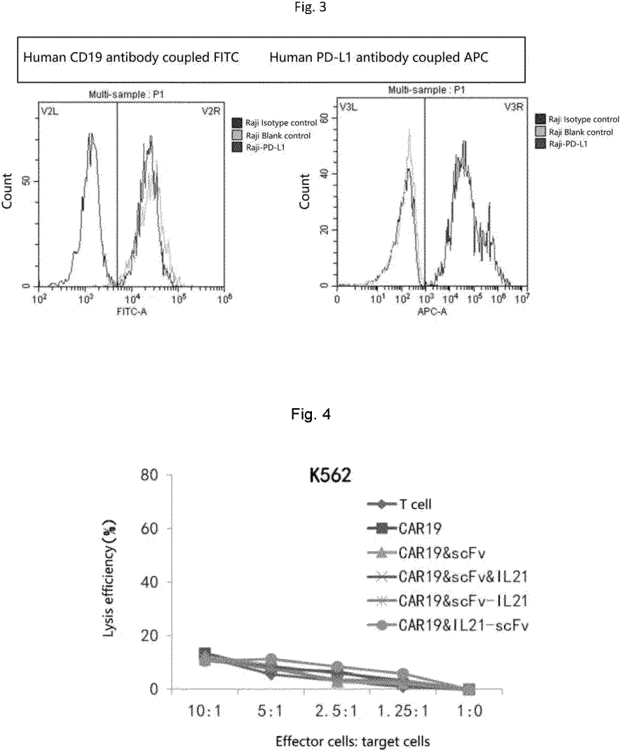

A lentivirus vector overexpression human PD-L1 is constructed, the lentivirus was packaged and concentrated, and then Raji cells were infected according to a proportion of MOI=10. After 72 hours of infection, the infection efficiency was detected with an APC anti-human PD-L1 antibody, positive cells were flow-sorted, and a Raji-PD-L1 overexpression target cell line was established by monoclonal cloning. Results were as shown in FIG. 3: the Raji cell line that overexpresses PD-L1 (clone number A3 is represented as Raji-PD-L1-A3) was successfully screened. Compared with Raji, the expression of a CD19 antigen in this Raji-PD-L1-A3 cell line is not changed.

### Embodiment 6: CART In Vitro Killing Function Evaluation

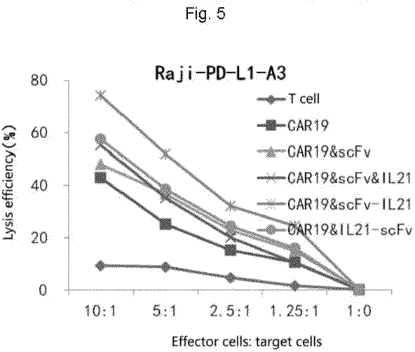

Killing experiment: each group of CAR-T and control T cells were labeled with carboxyfluorescein succinimidyl ester (CFSE) dye, and then co-cultured with the RAJI-PD-L1-A3 cell line according to the effect target ratio of 10:1, 5:1, 2.5:1, 1.25:1 and 1:0. After 4 hours, a killing supernatant was collected and retained for cytokine detection. Killed cells were stained with propidium iodide (PI) and Annexin V, and a CFSE negative RAJI-PD-L1-A3 cell group was selected. the late and early apoptosis of the RAJI-PD-L1-A3 cells were detected trough the flow cytometry; and a K562 cell with CD19 negative expression was used as a killing control. Results were as shown in FIG. 4 and FIG. 5: each experimental group does not have an apparent killing function on the K562 cell with the CD19 negative expression, but each group of CART has the apparent killing function on RAJI-PD-L1-A3 with CD19 positive expression, and CAR19&scFv-IL21 has the best killing effect. While the effect target ratio is 10:1, the efficiency of CAR19&scFv-IL21 killing RAJI-PD-L1-A3 is 74.33%.

### Embodiment 7: Cytokine Release in Process of CART Killing RAJI-PD-L1-A3

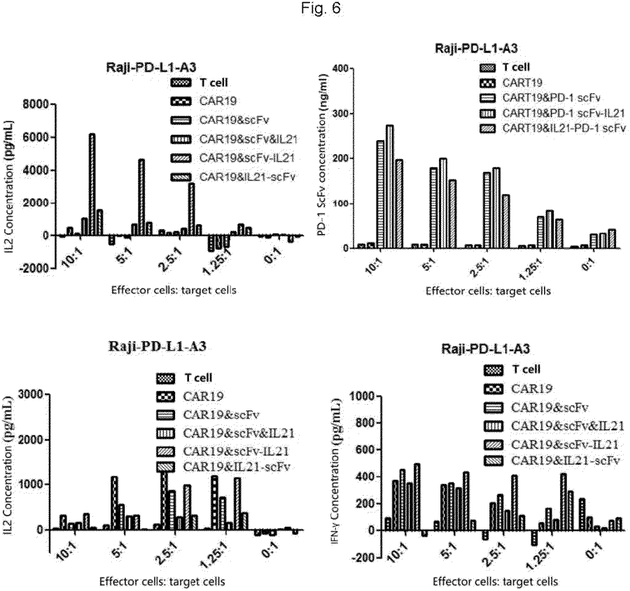

Factor detection: expression conditions of cytokines were detected according to Invitrogen IL2 detection instructions (catalog number: 88-7025), IL21 detection instructions (catalog number: 88-8218) and IFN-γ (catalog number: 88-7316), and a detection method of a PD-1 single-chain antibody was developed by our company. Molecule release results of the cytokine are as shown in FIG. 6: Compared with other groups, the process of CAR19&scFv-IL21 killing RAJI-PD-L1-A3 releases more IL21, PD-1 single-chain antibody (scFv) and IFN-γ, and the release of more IL2 suggests that CAR19&scFv-IL21 may play a killing role by overexpressing the above factor.

### Embodiment 8: CART In Vivo Efficacy Evaluation

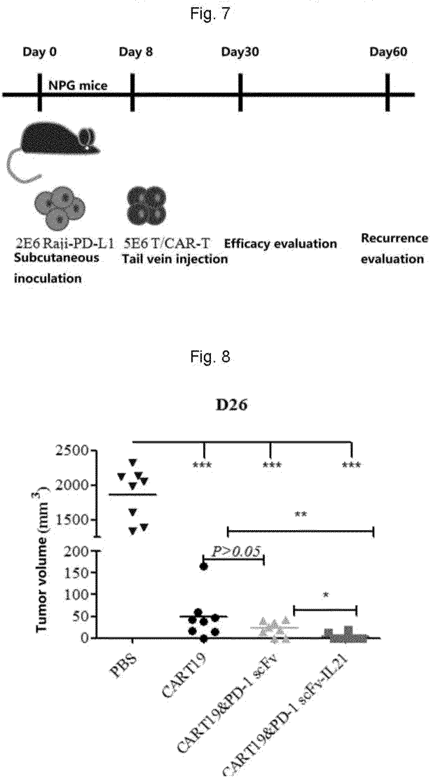

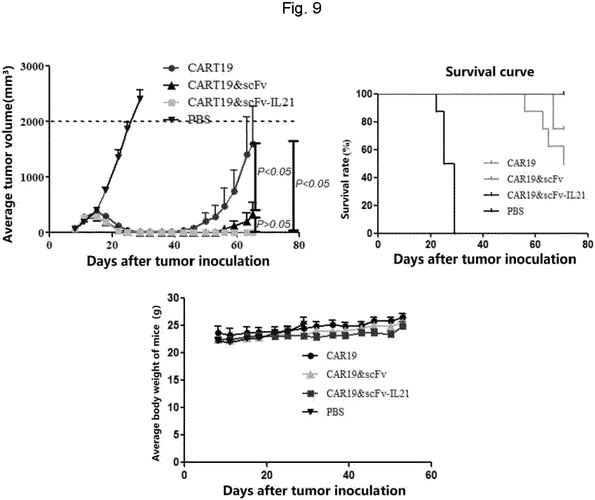

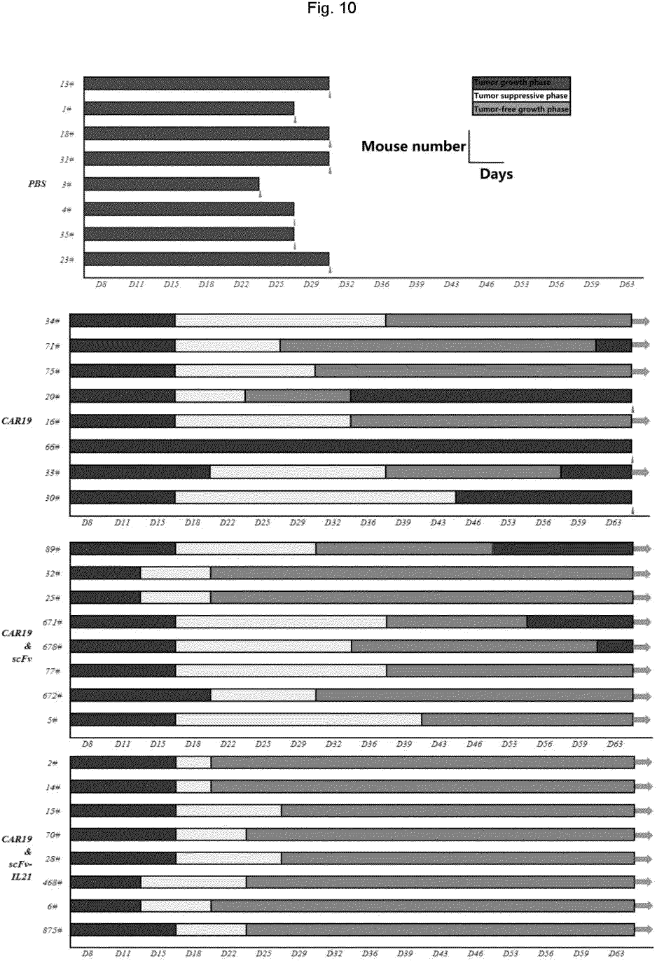

Experiment scheme: 5-week old female NPG mice were selected, and on the 0th day (Day 0), 2E5 Raji-PD-L1-A3/mouse was inoculated at subcutaneously, and there were a total of 40 mice; on the 7th day (DAY 7), the mice with a tumor volume of 100 mm³ were selected and grouped for an experiment, there were four groups in total, the tumor volume of each group was kept consistent as much as possible, and the mice with too large or too small tumor volume were excluded; on the 8th day (DAY 8), corresponding CART (CAR19, CAR19&scFv and CAR19&scFv-IL21) was injected into tail veins of the mice in each group, and phosphate buffered saline (PBS) was injected in a control group; and the tumor volume and weight were measured on Wednesday after administration, the tumor remission rate was evaluated while the control mice reach the tumor measurement endpoint, and the tumor recurrence rate and survival curve were evaluated two months later. The time axis of the experiment was as shown in FIG. 7. Each NPG mouse on D0 was inoculated with Raji-PD-L1 cells subcutaneously according to an amount of 2E6, the tumor volumes of the mice were measured on D7 and grouped, there were 8 mice in each group, CART cells were injected into the tail veins of the mice on D8 according to an amount of 5E6, the CART efficacy was evaluated within one month after tumor inoculation, and the ability of a drug to control recurrence was evaluated two months later. The average tumor volume, tumor remission rate and tumor inhibition rate of each group on D26 were as shown in Table 2 and FIG. 8. The average tumor volume of the CART19&scFv-IL21 group is significantly different from that of the CART19&scFv group, and is extremely significantly different from that of the CART19 group. The average recurrent tumor volume and recurrence rate of each group on D65 were as shown in FIG. 9 and Table 3. The average recurrent tumor volumes of the CART19&scFv-IL21 group and the CART19&scFv group are significantly different from that of the CART19 group, but there is no significant difference between the CART19&scFv-IL21 group and the CART19&scFv group. The individual tumor remission and recurrence conditions of the mice in the groups were as shown in FIG. 10, and the mice in the CART19&scFv-IL21 group have a longer tumor-free growth period.

TABLE 2

| Group | Average tumor volume (mm³) D26 | Complete remission rate (CRR) | Tumor inhibition rate (TGI, %) | P value^{$} |
|---|---|---|---|---|
| CART19 | 47.93 | 1/8 | 97.45% | <0.001 |
| CART19&PD-1 scFv | 22.03 | 2/8 | 98.82% | <0.001 |
| CART19&PD-1 scFv-IL21 | 3.889 | 6/8 | 99.79% | <0.001 |
| PBS | 1866 | 0/10 | - | - |

$: The average tumor volume of the CAR-T group was compared with that of the PBS group, T detection (Mann-Whitney).
*P<0.05, **P<0.01, and ***P<0.001.

TABLE 3

| | | | |
|---|---|---|---|
| Group | Average recurrent tumor volume (mm³) D65 | Recurrence rate (%) | P value^{$} |
| CART19 | 935.9858 | 5/8 (62.5%) | - |
| CART19 & scFv | 324.3721 | 3/8 (37.5%) | <0.05 |
| CART19 & scFv-IL21 | 0 | 0/8 (0%) | <0.05 |
| PBS | - | - | - |

$: Except the PBS group, the average recurrent tumor volume of each group was compared with that of the CART19 group, T detection (Mann-Whitney).
*P<0.05, **P<0.01, and ***P<0.001.

### Embodiment 9: Construction and Generation of T Cell Targeting Chimeric Antigen Receptor of Mesothelin (MSLN)

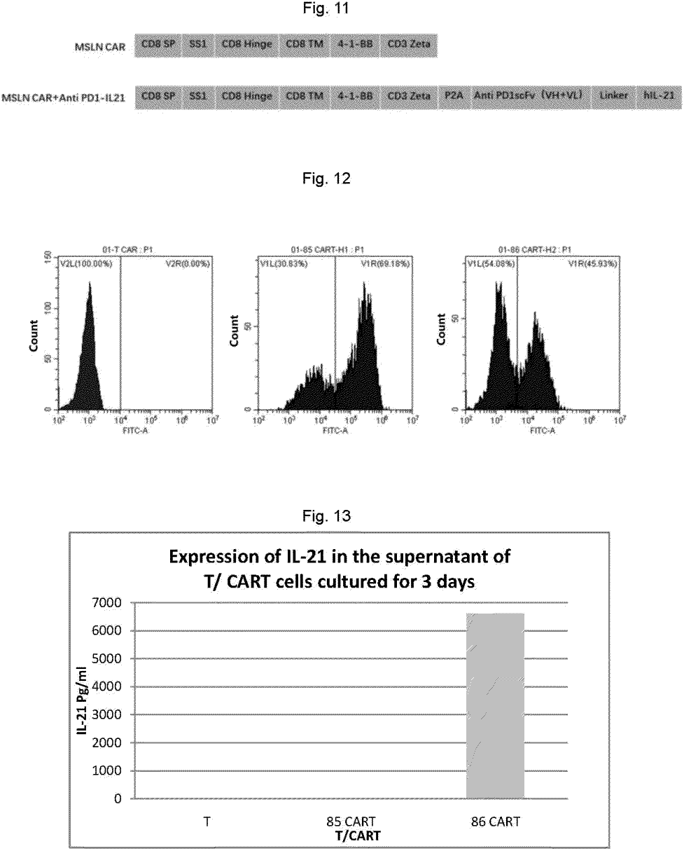

The following nucleotide sequences were synthesized on the basis of genes in FIG. 11, and according to a digestion site of a lentivirus vector, a nucleotide fragment was constructed onto the lentivirus vector, primers were designed, and the correctness of the vector construction was verified by sequencing results, herein SS1 was a scFv sequence of an anti-human MSLN antibody, a MSLN CAR plasmid was named as PCDHF85, and a MSLN CAR+Anti PD1-IL-21 plasmid was named as PCDHF86.

Packaging of lentivirus: the lentivirus was respectively packaged with the above two plasmids, and a packaging system was as shown in Table 4 below.

TABLE 4

| | | |
|---|---|---|
| | PCDHF-85 | PCDHF-86 |
| PCDH D (PMD2.G) | 24 µg | 36 µg |
| PCDH M (pMDLg/pRRE) | 24 µg | 36 µg |
| PCDH N (pRSV-Rev) | 24 µg | 36 µg |
| PCDHF85 | 48 µg | |
| PCDHF86 | | 72 µg |
| PEI (Polysciences 636951) | 240 µg | 360 µg |
| OPTI-MEMI (Gibco 31985070) | 12 mL | 18 mL |

5E6 293T cells were inoculated into a 10 cm cell culture dish, 10 mL of DMEM containing 10% FBS (DMEM Gibco, 11995040-1L; and FBS Gibco, 10091-148) was added, and it was cultured in a CO₂ incubator under conditions of 5% CO₂ and 37° C. for 24 h; and the lentivirus packaging was performed as shown in Table 4. CART cell preparation: PBMC cells were separated from blood (50 mL of the blood donated by No. 0068 volunteer of staff in Fapon Biotech) with Ficoll lymphocyte separation solution (Dakewe, AS1114546), and coupled with magnetic beads of a CD3/CD28 antibody (Dynabeads, CD3/CD28 CTS, catalog number: 40203D, and batch number: A2-011710E) to obtain the T cells separated throught a positive selection method, and the T cells were infected with the lentivirus according to MOI=5:1 to prepare CART cells. After 7 days of culture of the CART cells, the CAR positive rate of the CART cells was determined by detecting the MSLN antibody expression of the CART cells, as shown in FIG. 12.

### Embodiment 10: MSLN CAR-T Supernatant Detection

CART cells secrete IL-21. Firstly, the expression level of IL-21 in a culture supernatant of the CART cells was detected, and the initial cell density was 2E5 cells/ml. After 72 hours of culture, the supernatant was collected. A IL-21 ELISA test kit (Invitrogen, batch number: 220657) was used, and results were as shown in FIG. 13. The results show that 86CART cells normally secrete IL-21 and PD1 antibodies.

### Embodiment 11: Detection of MSLN CAR-T Tumor Cell Killing and Cytokine Release

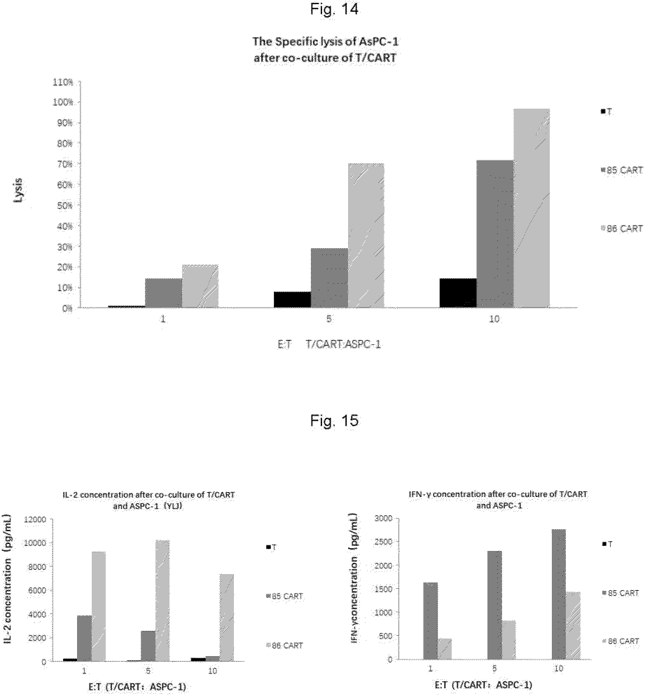

CART cells kill tumor cells. 2E4/100 ul/well AsPC-1 cells (ATCC, CRL-1682, Mesothelin expression positive cells) were respectively added to a 96-well flat bottom plate (Costar, catalog number: 25719016), the medium was RPMI1640 (Gibco, batch number: 2215748) + 10% FBS (Gibco, batch number: 2152441P), and it was cultured at 5% CO₂ and 37° C. for 48 h. T cells, 85CART cells and 86CART cells were taken respectively, and after being counted, the T cells were used to dilute the 85CART cells to a CAR+ positive rate of 45%. The cells were respectively added into each well according to a proportion of effector cells (CAR+CART cells): target cells=1:1, 5:1, and 10:1. The culture medium of the CART cells was X-VIVO 15. At 100 ul/well, the CART cells and tumor cells were mixed and cultured for 16 h, a supernatant was sucked for standby, the cells were gently washed with Dulbecco’s phosphate buffered saline (DPBS) (Hyclone, batch number: AE29431662) twice and then DPBS was sucked and removed. The suspended cells in the wells were further sucked and removed, then RPMI1640+10% FBS (100 ul) was added, and prepared Promega (CellTiter Glo Luminescent Cell Viability Assay, article number: 0000453271) substrate and buffer mixed solution were added at 100 ul/well. After 10 min, a multi-functional microplate reader (MOLECLAR DEVICES, SpectraMax i3X) was used to detect the fluorescence value of each well, and the fluorescence value of each well was compared with that of a AsPC-1 cell well (without adding the T/CART cells), thereby the ratio of living cells to apoptotic cells in each well was calculated. Detection results were as shown in FIG. 14, and the results show that for the killing effect of the AsPC-1 cells, 86CART>85CART>T.

A IL-2/IFN-γ ELISA kit (Human IL-21 Uncoated ELISA Kit, R&D, batch number: 223086-004; and IFN gamma Human Uncoated ELISA Kit, R&D, batch number: 223086-003) was used to detect the secretion levels of IL-2 and IFN-γ in the supernatant after CART and AsPC-1 cells were co-cultured, and detection results were as shown in FIG. 15. The results show that while the CART cells are co-cultured with the AsPC-1 cells, IL-2 and IFN-γ are normally secreted.

In descriptions of this description, descriptions of reference terms such as “one embodiment”, “some embodiments”, “examples”, “specific examples”, or “some examples” means that specific features, structures, materials, or characteristics described in combination with this embodiment or example are included in at least one embodiment or example of the present disclosure. In this description, the illustrative statements of the above terms unnecessarily refer to the same embodiments or examples. Furthermore, the specific features, structures, materials, or characteristics described may be combined in an appropriate mode in any one or more embodiments or examples. In addition, those skilled in the art may integrate and combine different embodiments or examples described in this description and the features of the different embodiments or examples in the case without conflicting.

Although the embodiments of the present disclosure are already shown and described above, it may be understood that the above embodiments are exemplary, and may not be understood as limitation to the present disclosure. Those of ordinary skill in the art may change, modify, replace and transform the above embodiments within the scope of the present disclosure.

What is claimed is:

1. A T lymphocyte, wherein the T lymphocyte co-expresses a fusion protein and a chimeric antigen receptor, wherein the chimeric antigen receptor comprises:
an extracellular region, wherein the extracellular region comprises a heavy chain variable region, a light chain variable region and a CD8 hinge region of a single chain antibody, and the single-chain antibody specifically recognizes a tumor antigen;
a transmembrane region, wherein the transmembrane region is connected with the extracellular region, the transmembrane region comprises a transmembrane segment of CD8 and is embedded into a cell membrane of the T lymphocyte; and
an intracellular region, wherein the intracellular region is connected to the transmembrane region, and the intracellular region comprises an intracellular segment of 4-1 BB and a CD3 ζ chain; and
the fusion protein comprises: an immune checkpoint single-chain antibody and a T cell activation molecule.

2. The T lymphocyte according to claim 1, wherein an immune checkpoint in the immune checkpoint single-chain antibody comprises at least one selected from PD-1, PD-L1, CTLA-4, TIM3, LAG3, BTLA and TIGIT; and
optionally, the T cell activation molecule comprises at least one selected from IL2, IL7, IL9, IL12, IL15, IL18 and IL21;
optionally, wherein the immune checkpoint is PD-1, and the T cell activation molecule is IL21;
optionally, wherein the C terminal of the IL21 is connected with the N terminal of a PD-1 single-chain antibody; preferably, the C terminal of the PD-1 single-chain antibody is connected with the N terminal of the IL21.

3-4. (canceled)

5. The T lymphocyte according to claim 1, wherein the fusion protein further comprises a linker peptide, and the linker peptide is arranged between the immune checkpoint single-chain antibody and the T cell activation molecule;
optionally, the linker peptide has an amino acid sequence shown in SEQ ID NO: 1; and
optionally, the N terminal of the linker peptide is connected with the C terminal of the immune checkpoint single-chain antibody, and the C terminal of the linker peptide is connected with the N terminal of the T cell activation molecule.

6. The T lymphocyte according to claim 1, wherein the fusion protein has an amino acid sequence shown in SEQ ID NO: 2 or SEQ ID NO: 3.

7. A lentivirus, wherein the lentivirus carries the following nucleic acid molecules:
(a) a nucleic acid molecule encoding a fusion protein, wherein the fusion protein comprises: an immune checkpoint single-chain antibody and a T cell activation molecule; and
(b) a nucleic acid molecule encoding a chimeric antigen receptor, wherein an extracellular region of the chimeric antigen receptor recognizes a tumor antigen.

8. The lentivirus according to claim 7, wherein an immune checkpoint in the immune checkpoint single-chain antibody is PD-1, the T cell activation molecule is IL21, and the fusion protein has an amino acid sequence shown in SEQ ID NO: 2 or 3; and
optionally, the tumor antigen is CD19, and the chimeric antigen receptor has an amino acid sequence shown in SEQ ID NO: 4;
optionally, wherein the nucleic acid molecule encoding the fusion protein has a nucleotide sequence shown in SEQ ID NO: 5 or 6; and
optionally, the nucleic acid molecule encoding the chimeric antigen receptor has a nucleotide sequence shown in SEQ ID NO: 7.

9. (canceled)

10. The lentivirus according to claim 7, wherein the lentivirus carries a nucleic acid molecule with a nucleotide sequence shown in SEQ ID NO: 8 or 9.

11. A transgenic lymphocyte, wherein the transgenic lymphocyte co-expresses a fusion protein and a chimeric antigen receptor, and the chimeric antigen receptor recognizes a tumor antigen,
wherein the chimeric antigen receptor comprises:
an extracellular region;
a transmembrane region, wherein the transmembrane region is connected with the extracellular region, and embedded into a cell membrane of the transgenic lymphocyte; and
an intracellular region, wherein the intracellular region is connected with the transmembrane region, and the intracellular region comprises an intracellular segment of an immune co-stimulatory molecule; and
the fusion protein comprises: an immune checkpoint single-chain antibody and a T cell activation molecule.

12. The transgenic lymphocyte according to claim 11, wherein the intracellular segment of the immune co-stimulatory molecule is independently selected from at least one of 4-1BB, OX-40, CD40L, CD27, CD30, CD28, CD3 and derivatives thereof;
optionally, the intracellular segment of the immune co-stimulatory molecule is an intracellular segment of 4-1BB and CD3;
optionally, the lymphocyte is a CD3⁺T lymphocyte;
optionally, the lymphocyte is a CD8⁺T lymphocyte;
optionally, the lymphocyte is a natural killer cell; and
optionally, the lymphocyte is a natural killer T cell.

13. The transgenic lymphocyte according to claim 11, wherein an immune checkpoint in the immune checkpoint single-chain antibody comprises at least one selected from PD-1, PD-L1, CTLA-4, TIM3, LAG3, BTLA and TIGIT;
optionally, the T cell activation molecule comprises at least one selected from IL2, IL7, IL9, IL12, IL15, IL18 and IL21;
optionally, the immune checkpoint is PD-1, and the T cell activation molecule is IL21;
optionally, the C terminal of the IL21 is connected with the N terminal of a PD-1 single-chain antibody;
optionally, the C terminal of the PD-1 single-chain antibody is connected with the N terminal of the IL21;
optionally, the fusion protein further comprises a linker peptide, and the linker peptide is arranged between the immune checkpoint single-chain antibody and the T cell activation molecule;
optionally, the linker peptide has an amino acid sequence shown in SEQ ID NO: 1;
optionally, the N terminal of the linker peptide is connected with the C terminal of the immune checkpoint single-chain antibody, and the C terminal of the linker peptide is connected with the N terminal of the T cell activation molecule; and
optionally, the fusion protein has an amino acid sequence shown in SEQ ID NO: 2 or SEQ ID NO: 3.

14. A construct, wherein the construct comprises:
a first nucleic acid molecule, wherein the first nucleic acid molecule encodes a fusion protein, and the fusion protein comprises:
an immune checkpoint single-chain antibody and a T cell activation molecule; and
a second nucleic acid molecule, wherein the second nucleic acid molecule encodes a chimeric antigen receptor, and the chimeric antigen receptor recognizes a tumor antigen,
wherein the fusion protein and the chimeric antigen receptor are as defined according to claim 1.

15. The construct according to claim 14, wherein the first nucleic acid molecule and the second nucleic acid molecule are set to express the fusion protein and the chimeric antigen receptor in a lymphocyte, and the fusion protein and the chimeric antigen receptor are in a non-fusion form;
optionally, further comprising:
a first promoter, wherein the first promoter is operably connected with the first nucleic acid molecule; and
a second promoter, wherein the second promoter is operably connected with the second nucleic acid molecule;
optionally, the first promoter and the second promoter are respectively independently selected from the promoter of U6, H1, CMV, EF-1, LTR or RSV;
optionally, further comprising:
an internal ribosome entry site sequence, wherein the internal ribosome entry site sequence is arranged between the first nucleic acid molecule and the second nucleic acid molecule, and the internal ribosome entry site has a nucleotide sequence shown in SEQ ID NO: 10;
optionally, further comprising:
a third nucleic acid molecule, wherein the third nucleic acid molecule is arranged between the first nucleic acid molecule and the second nucleic acid molecule, and the third nucleic acid molecule encodes a cleavable linker peptide, the cleavable linker peptide can be cleaved in the lymphocyte; and
optionally, the cleavable linker peptide has an amino acid sequence shown in SEQ ID NO: 11.

16. (canceled)

17. The construct according to claim 14, wherein a vector of the construct is a non-pathogenic virus vector; and
optionally, the virus vector comprises at least one selected from a retrovirus vector, a lentivirus vector and an adenovirus-related virus vector.

18. A method for preparing the T lymphocyte according to claim 1, comprising:
introducing a construct into a lymphocyte or a T lymphocyte, wherein the construct comprises:
a first nucleic acid molecule, wherein the first nucleic acid molecule encodes a fusion protein, and the fusion protein comprises:
an immune checkpoint single-chain antibody and a T cell activation molecule; and
a second nucleic acid molecule, wherein the second nucleic acid molecule encodes a chimeric antigen receptor, and the chimeric antigen receptor recognizes a tumor antigen,
wherein the fusion protein and the chimeric antigen receptor are as defined according to claim 1.

19. A therapeutic composition for treating a cancer, comprising:
the T lymphocyte according to claim 1.

20. The therapeutic composition according to claim 19, wherein the cancer comprises at least one selected from a liver cancer, a pancreatic cancer, an ovarian cancer, a bile duct cancer, a lung cancer, a stomach cancer, an intestinal cancer, an esophageal cancer and a breast cancer.

21-22. (canceled)

23. A method for treating the cancer, wherein the method comprises administering at least one of the followings to a subject suffering from the cancer:
the T lymphocyte according to claim 1; or
the therapeutic composition comprising the T lymphocyte according to claim 1.

24. The method according to claim 23, wherein the cancer comprises at least one selected from a liver cancer, a pancreatic cancer, an ovarian cancer, a bile duct cancer, a lung cancer, a stomach cancer, an intestinal cancer, an esophageal cancer and a breast cancer.

25-26. (canceled)

27. A method for reducing expression of an immune checkpoint on the surface of a T lymphocyte, comprising:
making the T lymphocyte express a chimeric antigen receptor and a fusion protein; or making the T lymphocyte co-culture with T cells expressing the chimeric antigen receptor and the fusion protein,
the fusion protein comprises: an immune checkpoint single-chain antibody and a T cell activation molecule,
wherein the chimeric antigen receptor and the fusion protein are as defined according to claim 1.

28. The method according to claim 27, wherein the immune checkpoint is PD-1, and the T cell activation molecule is IL21.


SEQUENCE LISTING (11 sequences)

SEQ ID NO: 1 - protein, 15 aa, Artificial Sequence
  Linker peptide
GGGGSGGGGSGGGGS

SEQ ID NO: 2 - protein, 390 aa, Artificial Sequence
  Fusion protein
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSGGGGSGGGGSGGGGSMHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 3 - protein, 390 aa, Artificial Sequence
  Fusion protein
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSGGGGSGGGGSGGGGSMEIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRGGGGSGGGGSGGGGSQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSS

SEQ ID NO: 4 - protein, 465 aa, Artificial Sequence
  Chimeric antigen receptor
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - DNA, 1170 bp, Artificial Sequence
  Nucleic acid molecule encoding fusion protein
gagatcgtgctgacccagtctccagccacactgagcctgtctcctggcgagagagccaccctgtcttgtagggccagccagtccgtgagctcttacctggcctggtatcagcagaagccaggccaggccccaagactgctgatctacgacgcctccaacagagccaccggcatcccagccagattttctggctccggctctggcaccgacttcacactgaccatcagctctctggagccagaggatttcgccgtgtattactgccagcagagctctaactggccaagaacattcgggcaggggaccaaggtggaaatcaagaggggcggcggcggctctggcggcggcggctccggcggcggcggctctcaggtgcagctggtggagagcggcggcggagtggtgcagccaggcagatctctgagactggattgcaaggccagcggcatcaccttcagcaattccggcatgcactgggtgcggcaggcccccggcaagggcctggagtgggtggccgtgatctggtatgacggctctaagcggtactatgccgactctgtgaagggcagattcaccatctccagggacaactccaagaataccctgttcctgcagatgaacagcctgagggccgaggataccgccgtgtactattgcgccaccaacgacgattactggggccagggcacactggtgaccgtgtccagcggcggcggcggctctggcggcggcggctccggcggcggcggctctatgcacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcc

SEQ ID NO: 6 - DNA, 1170 bp, Artificial Sequence
  Nucleic acid molecule encoding fusion protein
cacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccggtggcggtggctcgggcggtggtgggtcgggtggcggcggatctatggagatcgtgctgacccagtctccagccacactgagcctgtctcctggcgagagagccaccctgtcttgtagggccagccagtccgtgagctcttacctggcctggtatcagcagaagccaggccaggccccaagactgctgatctacgacgcctccaacagagccaccggcatcccagccagattttctggctccggctctggcaccgacttcacactgaccatcagctctctggagccagaggatttcgccgtgtattactgccagcagagctctaactggccaagaacattcgggcaggggaccaaggtggaaatcaagaggggcggcggcggctctggcggcggcggctccggcggcggcggctctcaggtgcagctggtggagagcggcggcggagtggtgcagccaggcagatctctgagactggattgcaaggccagcggcatcaccttcagcaattccggcatgcactgggtgcggcaggcccccggcaagggcctggagtgggtggccgtgatctggtatgacggctctaagcggtactatgccgactctgtgaagggcagattcaccatctccagggacaactccaagaataccctgttcctgcagatgaacagcctgagggccgaggataccgccgtgtactattgcgccaccaacgacgattactggggccagggcacactggtgaccgtgtccagc

SEQ ID NO: 7 - DNA, 1395 bp, Artificial Sequence
  Nucleic acid molecule encoding chimeric antigen receptor
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 8 - DNA, 2622 bp, Artificial Sequence
  Nucleic acid molecule carried by a lentivirus
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccactaacttctccctgttgaaacaagcaggggatgtcgaagagaatcccgggccagagatcgtgctgacccagtctccagccacactgagcctgtctcctggcgagagagccaccctgtcttgtagggccagccagtccgtgagctcttacctggcctggtatcagcagaagccaggccaggccccaagactgctgatctacgacgcctccaacagagccaccggcatcccagccagattttctggctccggctctggcaccgacttcacactgaccatcagctctctggagccagaggatttcgccgtgtattactgccagcagagctctaactggccaagaacattcgggcaggggaccaaggtggaaatcaagaggggcggcggcggctctggcggcggcggctccggcggcggcggctctcaggtgcagctggtggagagcggcggcggagtggtgcagccaggcagatctctgagactggattgcaaggccagcggcatcaccttcagcaattccggcatgcactgggtgcggcaggcccccggcaagggcctggagtgggtggccgtgatctggtatgacggctctaagcggtactatgccgactctgtgaagggcagattcaccatctccagggacaactccaagaataccctgttcctgcagatgaacagcctgagggccgaggataccgccgtgtactattgcgccaccaacgacgattactggggccagggcacactggtgaccgtgtccagcggcggcggcggctctggcggcggcggctccggcggcggcggctctatgcacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcc

SEQ ID NO: 9 - DNA, 2622 bp, Artificial Sequence
  Nucleic acid molecule carried by a lentivirus
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccactaacttctccctgttgaaacaagcaggggatgtcgaagagaatcccgggccacacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgttttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattccggtggcggtggctcgggcggtggtgggtcgggtggcggcggatctatggagatcgtgctgacccagtctccagccacactgagcctgtctcctggcgagagagccaccctgtcttgtagggccagccagtccgtgagctcttacctggcctggtatcagcagaagccaggccaggccccaagactgctgatctacgacgcctccaacagagccaccggcatcccagccagattttctggctccggctctggcaccgacttcacactgaccatcagctctctggagccagaggatttcgccgtgtattactgccagcagagctctaactggccaagaacattcgggcaggggaccaaggtggaaatcaagaggggcggcggcggctctggcggcggcggctccggcggcggcggctctcaggtgcagctggtggagagcggcggcggagtggtgcagccaggcagatctctgagactggattgcaaggccagcggcatcaccttcagcaattccggcatgcactgggtgcggcaggcccccggcaagggcctggagtgggtggccgtgatctggtatgacggctctaagcggtactatgccgactctgtgaagggcagattcaccatctccagggacaactccaagaataccctgttcctgcagatgaacagcctgagggccgaggataccgccgtgtactattgcgccaccaacgacgattactggggccagggcacactggtgaccgtgtccagc

SEQ ID NO: 10 - DNA, 588 bp, Artificial Sequence
  Nucleotide sequence of the internal ribosome entry site
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 11 - protein, 21 aa, Artificial Sequence
  Cleavable linker peptide
IDATNFSLLKQAGDVEENPGP